(19) United States
(12) Patent Application Publication
(10) Pub. No.: US 2026/0083522 A1
(43) Pub. Date: Mar. 26, 2026

(54) CATHETER ROBOT AND HUMAN-MACHINE INTERFACE FOR CONTROLLING A MODULE FOR DRIVING AN ELONGATE FLEXIBLE MEDICAL INSTRUMENT

(71) Applicant: ROBOCATH, ROUEN (FR)

(72) Inventors: Benoît HOEFLER, ROUEN (FR); Arthur CAHAREL, ROUEN (FR); Gaëtan GIGLEUX, LYON (FR); Bruno FOURNIER, SAINT OUEN (FR)

(73) Assignee: ROBOCATH, ROUEN (FR)

(21) Appl. No.: 19/107,850

(22) PCT Filed: Aug. 25, 2023
(86) PCT No.: PCT/EP2023/073433

§ 371 Date: Feb. 28, 2025

(30) Foreign Application Priority Data
Sep. 1, 2022 (FR) FR2208803

(51) Int. Cl. A61B 34/37; A61B 34/00; A61B 34/30

(52) U.S. Cl. CPC A61B 34/37; A61B 34/76; A61B 34/77; A61B 2034/301

(57) ABSTRACT

A catheter robot including a drive module for an elongated flexible medical instrument, in translation along a main elongation axis of the elongated flexible medical instrument and in rotation around the main elongation axis of the elongated flexible medical instrument, simultaneously or alternatively, a human-machine interface for controlling the drive module. The human-machine interface is structured to, in a first mode of operation, control the translation drive of said elongated flexible medical instrument in speed, and control the rotation drive of said elongated flexible medical instrument in position.

### FIELD OF THE INVENTION

The invention relates to a catheter robot and a human-machine interface for controlling a module for driving an elongated flexible medical instrument.

### BACKGROUND OF THE INVENTION

The introduction of an elongated flexible medical device into a blood vessel, artery, or vein of a patient is generally monitored under X-rays. To avoid exposing the surgeon or another practitioner performing this introduction to excessive X-rays, it is known to use a catheter robot equipped with a module for driving the elongated flexible medical instrument, allowing the practitioner to remotely manipulate the elongated flexible medical instrument, thus performing tele-diagnosis or tele-intervention. The driving module transmits to the elongated flexible medical instrument introduced into the blood vessel a translational movement and/or a rotational movement that can be combined together.

Translational movement allows the elongated flexible medical instrument to move back and forth inside the blood vessel in which it is introduced. Rotational movement facilitates these movements of the elongated flexible medical instrument even in areas of the blood vessel that are highly stenosed or have significant tortuosity, as well as when passing branches between blood vessels.

The translational and rotational movements of the elongated flexible medical instrument are usually controlled by a mobile control organ operated by the practitioner during the intervention on the patient.

According to a first prior art, the control organ is configured to control, in position, translational and rotational movements of the elongated flexible medical instrument. When the movements of the elongated flexible medical instrument are controlled in position, a displacement of the control organ causes a displacement of the elongated flexible medical instrument with an amplitude proportional to the amplitude of displacement of the control organ. A disadvantage of this first prior art is that the translational movement, although precise, can become too slow at certain times, particularly at the beginning of the introduction of the catheter into the patient, when the catheter must reach the specific area of the patient's blood system where it will be used.

According to a second prior art, the control organ is configured to control, in speed, the translational and rotational movements of the elongated flexible medical instrument. When the movements of the elongated flexible medical instrument are controlled in speed, the displacement of the control organ causes a displacement of the elongated flexible medical instrument at a speed proportional to the amplitude of displacement of the control organ. A disadvantage of this second prior art is that the rotational movement, although fast, can lack precision at certain times, particularly when passing branches when the catheter is reaching the specific area of the patient's blood system where it will be used.

It is necessary to obtain a mode of driving the elongated flexible medical instrument that is both sufficiently precise and sufficiently fast, at least in most usage situations, while offering the practitioner good ergonomics for piloting this elongated flexible medical instrument.

The realization of tests, with a panel of practitioners, taking into account most of the usual usage situations of the elongated flexible medical instrument, surprisingly revealed that the configuration considered to achieve the best overall compromise between, on the one hand, driving efficiency and, on the other hand, piloting ergonomics, is a configuration in which:
the translational drive of the elongated flexible medical instrument is controlled in speed,
while the rotational drive of the elongated flexible medical instrument is controlled in position.

On the one hand, the position control of the rotation of the elongated flexible medical instrument allows it to be turned around its axis with a rotation angle proportional to the rotation amplitude of the control organ, which allows the practitioner to intuitively turn the elongated flexible medical instrument by a precise rotation angle.

On the other hand, the speed control of the translation of the elongated flexible medical instrument allows it to move inside the corresponding blood vessel at a speed proportional to the displacement amplitude of the control organ, which allows the elongated flexible medical instrument to move over a long distance with a compact control organ.

Moreover, thanks to the speed control, it is possible to adapt the translation speed of the elongated flexible medical instrument according to the area of the blood vessel it crosses. For example, to cross curved areas of the vessel, the translation speed of the elongated flexible medical instrument can be reduced to avoid damaging the vessel wall by a collision between the elongated flexible medical instrument and this wall. Therefore, the present invention allows, thanks to the combination of the speed control of the translation of the elongated flexible medical instrument and the position control of the rotation of the elongated flexible medical instrument, to obtain a catheter robot presenting a better compromise between good precision achieved and great ease of use by the practitioner, resulting in an efficient and ergonomic catheter robot.

### OBJECTS OF THE INVENTION

The purpose of the present invention is to provide a catheter robot and a human-machine interface for controlling a module for driving an elongated flexible medical instrument that at least partially overcomes the disadvantages of the aforementioned prior arts.

According to the invention, a catheter robot comprises:
a driving module for an elongated flexible medical instrument, in translation along a main elongation axis of said elongated flexible medical instrument and in rotation around the main elongation axis of said elongated flexible medical instrument, simultaneously or alternatively,
a human-machine interface for controlling said driving module,

characterized in that said human-machine interface is structured in such a way that, in a first mode of operation:
controlling, in speed, the translational drive of said elongated flexible medical instrument,
controlling, in position, the rotational drive of said elongated flexible medical instrument.

As explained above, thanks to the combination of the speed control of the translation of the elongated flexible medical instrument and the position control of the rotation of the elongated flexible medical instrument, the obtained catheter robot presents a better compromise between good precision achieved and great ease of use by the practitioner, resulting in an efficient and ergonomic catheter robot.

According to preferred embodiments, the invention includes one or more of the following features that can be used separately or in partial combination with each other or in total combination with each other, with the aforementioned object of the invention.

Preferably, said elongated flexible medical instrument is a catheter guide, and/or said elongated flexible medical instrument is a guiding catheter or a micro-catheter.

Preferably, said human-machine interface includes a mobile control organ intended to be manipulated by the hand of a user, and which is structured in such a way that, in the first mode of operation:
a translational displacement of said mobile control organ with a translation amplitude causes a translational displacement of said elongated flexible medical instrument with a speed proportional to said translation amplitude,
a rotational displacement of said mobile control organ with a rotation amplitude causes a rotational displacement of said elongated flexible medical instrument with a rotation angle proportional to said rotation amplitude.

Thus, an elaborate kinematics of simultaneous translational and rotational displacement can be achieved by a simple and robust structure of the drive organ.

Preferably, said human-machine interface is structured to control, in position, the rotational drive of said elongated flexible medical instrument, with a proportionality coefficient between, on the one hand, the rotational displacement of said control organ and, on the other hand, the rotational displacement of said elongated flexible medical instrument, said proportionality coefficient being modifiable by a selection of the user of the catheter robot.

Preferably, said human-machine interface for control includes:
a control organ for driving, in translation and rotation, said elongated flexible medical instrument,
a safety element, allowing to block or unblock the drive of said elongated flexible medical instrument by said control organ for driving.

The control organ is inherently sensitive. Thanks to the safety element, an inadvertent triggering by the control organ of the drive of the elongated flexible medical instrument can be more easily avoided. Preferably, the safety element at least prevents the inadvertent triggering of the translational drive of the elongated flexible medical instrument. This translation being controlled in speed, there is a greater risk (than for a position control) for the patient's health in case of inadvertent triggering.

Preferably, said safety element includes a safety surface capable of detecting the contact or pressure of the hand of a user in order to unblock said control organ for driving, said safety surface being preferably a touch surface, or a capacitive touch surface, or a capacitive touch surface covered with a coating including titanium, or a capacitive touch surface covered with titanium paint.

The safety element thus presents both simplicity of use and high operational efficiency.

Preferably, said human-machine interface for control includes haptic feedback for only the translation of the elongated flexible medical instrument, preferably in the form of vibrations, or preferably in the form of vibrations whose frequency is proportional to the translation speed of the elongated flexible medical instrument.

The user of the catheter robot thus receives feedback on the translational drive he has commanded, which allows him to verify that this drive is indeed taking place as he wished. When the vibrations have a frequency proportional to the translation speed of the elongated flexible medical instrument, it is possible to detect the passage of the elongated flexible medical instrument in tortuous or stenosed areas, which allows the user to know when it is appropriate to modify or adapt the command of the translational drive of the elongated flexible medical instrument and/or to command the rotation of the elongated flexible medical instrument.

Preferably, the human-machine interface is structured in such a way that, in a second mode of operation:
it controls, in speed, the translational drive of said elongated flexible medical instrument,
it controls, in speed, the rotational drive of said elongated flexible medical instrument.

Preferably, said human-machine interface for control includes a rod:
which is translatable by the hand of a user in order to drive said elongated flexible medical instrument in translation,
which is rotatable by the hand of a user in order to drive said elongated flexible medical instrument in rotation,
and which preferably includes a touch surface.

The rod preferably has an elongated shape like the elongated flexible medical instrument. This makes the control of the drive of the elongated flexible medical instrument more ergonomic.

Preferably, the rod comprises two parts sliding into each other:
a first part that is mobile in translation and rotation and is intended to be manipulated by the hand of a user,
a second part that is coupled in rotation with the first part and is decoupled in translation from the first part to remain fixed in translation.

The two parts of the rod sliding into each other, and the second part of the rod being decoupled in translation from the first part, the overall size of the human-machine interface can be reduced while ensuring effective control of the translation and rotation drive of the elongated flexible medical instrument.

Preferably, the human-machine interface also includes a rotating ring disposed around the rod, the rotation angle of the rotating ring around the rod being representative of the selected rotation speed for the elongated flexible medical instrument when controlling the rotation drive of the elongated flexible medical instrument in speed.

Preferably, the human-machine interface also includes a return elastic element, in the rest position, of the rotating ring disposed around the rod, the return elastic element preferably comprising a return spring.

The rest position corresponds to the position of the rotating ring before its rotation. In other words, in the rest position, the rotation speed of the elongated flexible medical instrument is zero. Also, thanks to the return elastic element, it is possible to stop the rotation drive of the elongated flexible medical instrument when the user stops actuating the rotating ring. Moreover, the return elastic element also allows reducing the overall size of the human-machine interface as it reduces the size and stroke of the rotating ring.

Preferably, the human-machine interface includes a crank that can be rotated by the hand of a user to drive the elongated flexible medical instrument in rotation.

Preferably, the human-machine interface also includes a return elastic element, in the rest position, for the translation drive only, of the mobile control organ or the rod, the return elastic element preferably comprising a return spring.

The rest position corresponds to the position of the mobile control organ or the rod before their translation. In other words, in the rest position, the translation speed of the elongated flexible medical instrument is zero. Also, thanks to the return elastic element, it is possible to stop the translation drive of the elongated flexible medical instrument when the user stops actuating the mobile control organ. Moreover, the return elastic element also allows reducing the overall size of the human-machine interface as it reduces the size and stroke of the mobile control organ.

The rotation angle of the control organ or the rod before their return to the rest position is thus preserved.

Preferably, the catheter robot also includes another drive module for another elongated flexible medical instrument, in translation along a main elongation axis of the other elongated flexible medical instrument and in rotation around the main elongation axis of the other elongated flexible medical instrument, the human-machine interface also controlling the other drive module and being structured to, in the first mode of operation:
control, in speed, the translation drive of the other elongated flexible medical instrument,
control, in position, the rotation drive of the other elongated flexible medical instrument.

The catheter robot can thus be equipped with two distinct medical instruments whose translation drive is controlled in speed and rotation drive is controlled in position. Additional functionalities can thus be added to the catheter robot. Moreover, the same human-machine interface allows controlling the drive of both medical instruments, which reduces the overall size of the catheter robot.

Preferably, the catheter robot includes at least one additional drive module, in translation, of an additional elongated flexible medical instrument, the additional elongated flexible medical instrument surrounding the elongated flexible medical instrument over part of its length, the human-machine interface also controlling the additional drive module, the human-machine interface being structured to control, in speed, the translation drive of the additional elongated flexible medical instrument.

The additional drive module being also controlled by the same human-machine interface as the first drive module, the overall size of the catheter robot is reduced. Moreover, thanks to the additional drive module and the additional elongated flexible medical instrument, it is possible to add additional functionalities to the catheter robot.

Preferably, the additional elongated flexible medical instrument is a catheter, preferably a stent or balloon catheter.

Preferably, the human-machine interface also includes a knob that can be rotated by the hand of a user to control in speed and drive in translation the additional elongated flexible medical instrument.

The knob has the advantage of being simple to use and having a particularly reduced size. Thus, for a simple control, such as the translation of the additional elongated flexible medical instrument, an improvement in the compromise between ease of use and the overall size of the human-machine interface of the catheter robot is achieved.

Preferably, the human-machine interface also includes an additional elastic return element for the knob in the rest position, which preferably comprises one or more additional return springs.

The rest position corresponds to the position of the knob before it is rotated. In other words, in the rest position, the translation speed of the additional elongated flexible medical instrument is zero. Also, thanks to the additional elastic return element, it is possible to stop the translation drive of the additional elongated flexible medical instrument when the user stops actuating the knob. Moreover, the additional elastic return element also allows reducing the overall size of the human-machine interface, as it reduces the size and stroke of the knob.

Preferably, the human-machine interface is structured to:
control only the translation drive of the elongated flexible medical instrument in speed,
control only the rotation drive of the elongated flexible medical instrument in position.

Preferably, the human-machine interface is structured, in a third mode of operation, to:
control the translation drive of the elongated flexible medical instrument in position, step by step, i.e., by moving a predetermined step with each impulse received by the human-machine interface,
control the rotation drive of the elongated flexible medical instrument in position, step by step, i.e., by moving a predetermined step with each impulse received by the human-machine interface.

According to another aspect, the invention relates to a human-machine interface for controlling a drive module of an elongated flexible medical instrument, in translation along a main elongation axis of the elongated flexible medical instrument and in rotation around the main elongation axis of the elongated flexible medical instrument, in a catheter robot, characterized in that it is structured to, in a first mode of operation:
control the translation drive of the elongated flexible medical instrument in speed,
control the rotation drive of the elongated flexible medical instrument in position.

Preferably, the human-machine interface includes a mobile control organ intended to be manipulated by the hand of a user, and which is structured so that, in the first mode of operation:
a translational movement of the mobile control organ with a translation amplitude causes a translational movement of the elongated flexible medical instrument with a speed proportional to the translation amplitude,
a rotational movement of the mobile control organ with a rotation amplitude causes a rotational movement of the elongated flexible medical instrument with a rotation angle proportional to the rotation amplitude.

Other features and advantages of the invention will appear from the following description of a preferred embodiment of the invention, given as an example and with reference to the appended drawings.

### BRIEF DESCRIPTION OF THE DRAWINGS

FIG. 1 schematically represents an example of a catheter robot according to an embodiment of the invention.

FIG. 2 represents a schematic longitudinal sectional view of an example of an elongated flexible medical instrument.

FIG. 3 is a schematic perspective view of an example of a human-machine interface of the catheter robot of FIG. 1 according to an embodiment of the invention.

FIG. 4 is a schematic perspective view of an example of a part of a mobile control organ, or drive control organ, of the human-machine interface of FIG. 3.

FIG. 5 is a schematic perspective view of an example of a mobile control organ or drive control organ of the human-machine interface of FIG. 3.

FIG. 6 represents a schematic side view of an example of a human-machine interface of the catheter robot of FIG. 1 according to another embodiment of the invention.

FIG. 7 represents a schematic side view of an example of a human-machine interface of the catheter robot of FIG. 1 according to another embodiment of the invention.

FIG. 8 schematically represents an example of a catheter robot according to another embodiment of the invention.

FIG. 9 is a schematic perspective view of an example of a human-machine interface of the catheter robot of FIG. 8 according to an embodiment of the invention.

### DETAILED DESCRIPTION OF THE EMBODIMENTS OF THE INVENTION

In the various figures, the same references designate identical or similar elements.

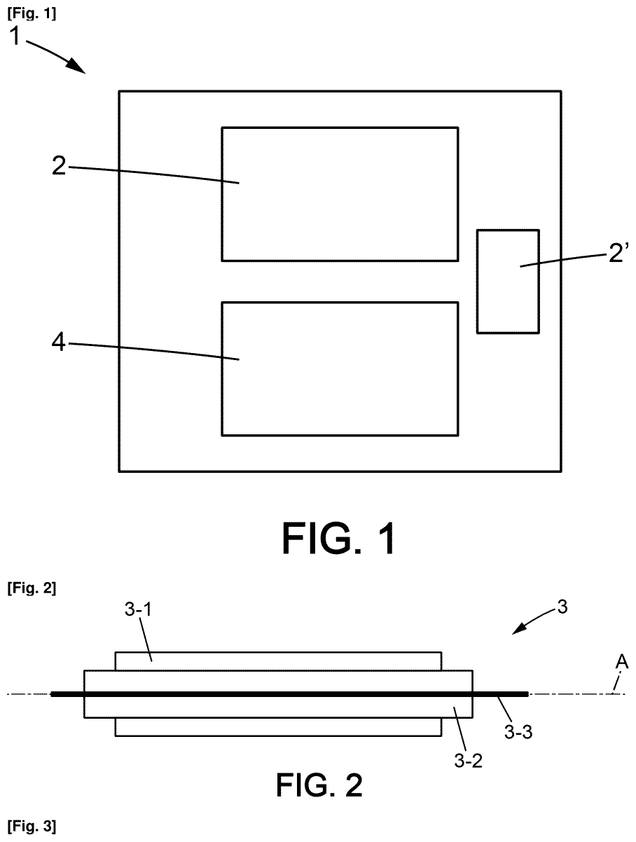

FIG. 1 schematically represents a catheter robot 1 according to a first embodiment of the invention. The catheter robot 1 includes a drive module 2 for an elongated flexible medical instrument 3 (illustrated in FIG. 2) and a human-machine interface 4 for controlling the drive module 2.

The elongated flexible medical instrument 3 can, for example, be an organ to be introduced into a patient's canal and moved within this canal, particularly an artery or vein of the patient. As illustrated in FIG. 2, the elongated flexible medical instrument 3 extends along an axis A, called the main elongation axis.

Preferably, the elongated flexible medical instrument 3 includes a catheter guide, and/or a guide catheter, and/or a micro-catheter. The elongated flexible medical instrument 3 can also or alternatively include a catheter, for example, of the balloon or stent type. In the non-limiting example of FIG. 2, the elongated flexible medical instrument 3 includes a guide catheter 3-1, a micro-catheter 3-2, and a catheter guide 3-3 coaxial with axis A. The guide catheter 3-1 and the micro-catheter 3-2 are hollow at least on a portion near the patient when the elongated flexible medical instrument 3 is introduced into the patient. The catheter guide 3-3 can be a solid metal wire. The diameter of the guide catheter 3-1 is larger than the diameter of the micro-catheter 3-2, and the diameter of the catheter guide 3-3 is smaller than the diameter of the micro-catheter 3-2. The catheter guide 3-3 is thus inserted, at least partially, into the micro-catheter 3-2, which is in turn inserted, at least partially, into the guide catheter 3-1.

Advantageously, the drive module 2 is structured to be connectable to the elongated flexible medical instrument 3. As will be detailed, the drive module 2 is configured to move the elongated flexible medical instrument 3 in translation along the main elongation axis A, and/or in rotation around the main elongation axis A.

In some cases, the catheter robot 1 may also include an additional drive module 2′ for an additional elongated flexible medical instrument.

Advantageously, the additional elongated flexible medical instrument surrounds the elongated flexible medical instrument 3, at least over a portion of the length of the elongated flexible medical instrument 3. Thus, the additional elongated flexible medical instrument is, for example, a catheter, preferably a stent or balloon catheter.

The additional drive module 2′ is structured to be connectable to the additional elongated flexible medical instrument. The additional drive module 2′ is configured to move the additional elongated flexible medical instrument in translation.

As will be detailed, the human-machine interface 4 is also configured to control the additional drive module 2′.

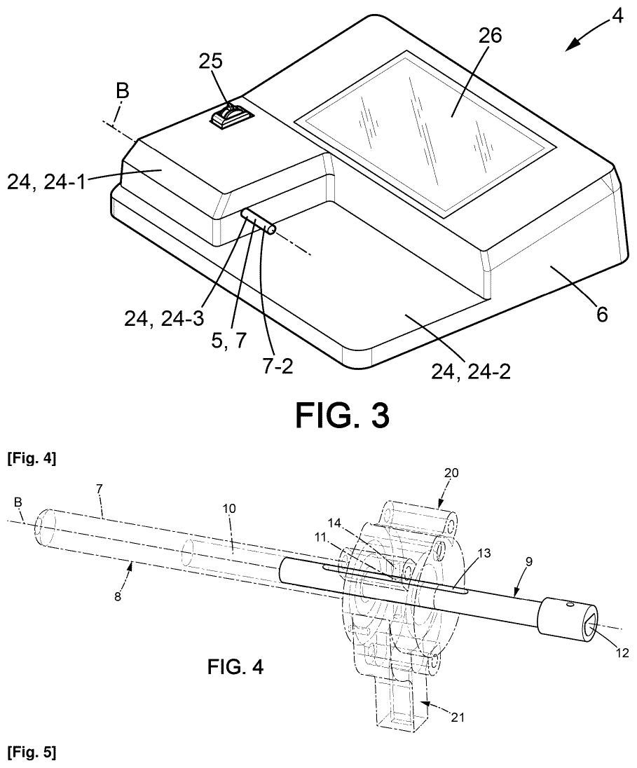

The human-machine interface 4 will now be described with reference to FIGS. 3 to 5.

The human-machine interface 4 includes a control organ 5 for the elongated flexible medical instrument 3. The control organ 5 is, for example, connected to housing 6 of the human-machine interface 4.

Preferably, control organ 5 is a mobile control organ intended to be manipulated by a user of the catheter robot 1. For example, control organ 5 can be manipulated by the user's hand. By “manipulate,” it is meant here that an effort is exerted by the user voluntarily on the control organ 5.

As will be detailed, the manipulation of the control organ 5 drives the elongated flexible medical instrument 3 in translation along the main elongation axis A and/or in rotation around this axis A. In particular, from the control organ 5, the translation of the elongated flexible medical instrument 3 along its main elongation axis A can be commanded in one direction or the other (advance or retreat). Similarly, from the control organ 5, the rotation of the elongated flexible medical instrument 3 around its main elongation axis A can be commanded in one direction or the other (clockwise or counterclockwise). Thus, control organ 5 is notably a control organ for driving, in translation and rotation, the elongated flexible medical instrument 3. As will be detailed, control organ 5 can allow the translation and/or rotation of the elongated flexible medical instrument 3 to be commanded from two types of commands: a position command or a speed command. These types of commands are described below.

Advantageously, control organ 5 has an elongated shape like the elongated flexible medical instrument 3. This makes the control of the drive of the elongated flexible medical instrument 3 more ergonomic.

In FIGS. 3 to 5, the mobile control organ 5 includes a rod 7 extending along a longitudinal axis B. The rod 7, for example, has a generally cylindrical shape.

In some cases, rod 7 is formed of a single piece. In other cases, rod 7 is formed by several distinct pieces connected together, as will be detailed later with reference to FIG. 4.

The rod 7 is partially introduced inside the housing 6 of the human-machine interface 4. Advantageously, a first end portion 7-1 of the rod 7 is inserted into the housing 6, with a second end portion 7-2 opposite the first end portion of the rod 7 being outside the housing 6. The user of the catheter robot 1 can thus manipulate the rod 7 by its second end portion 7-2.

The manipulation of the rod 7 can include moving the rod 7 or a part of it in translation along its longitudinal axis B in one direction or the other (advance or retreat). The manipulation of the rod 7 can also include moving the rod 7 or a part of it in rotation around this longitudinal axis B in one direction or the other (clockwise or counterclockwise). As will be explained below, the rod 7 is structured so that the movement of the rod 7 (or a part of it) in translation along the axis B or in rotation around the axis B commands a movement of the elongated flexible medical instrument 3 in, respectively, translation along its main elongation axis A or rotation around the main elongation axis A.

In the non-limiting embodiment illustrated in FIG. 4, rod 7 includes a first part 8 and a second part 9. The first part 8 and the second part 9 form two distinct pieces.

The first part 8 and the second part 9 each include a lateral wall, preferably substantially cylindrical.

The first part 8 of the rod 7 is hollow at least at one end portion, so as to form a cavity 10 extending substantially parallel to the axis B and delimited by the lateral wall of the first part 8.

At least one hole 11, for example circular, passes through the lateral wall of the first part 8 between the outside of the rod 7 and the cavity 10. Advantageously, two holes 11 arranged opposite each other perpendicularly to the axis B pass through the lateral wall of the first part 8.

The second part 9 is preferably hollow, so as to form a cavity 12 extending substantially parallel to the axis B and delimited by the lateral wall of the second part 9.

Preferably, a cross-section of the second part 9 is smaller than the cross-section of the cavity 10 of the first part 8. The second part 9 can thus be introduced, at least partially, into the cavity 10. Advantageously, there is a clearance between the lateral wall of the first part 8 and the lateral wall of the second part 9 when the second part 9 is introduced into the first part 8.

As shown in FIG. 4, the second part 9 includes at least one slot 13 passing through the lateral wall of the second part 9 between the outside of the rod 7 and the cavity 12. Advantageously, two slots 13 arranged opposite each other perpendicularly to the axis B pass through the lateral wall of the second part 9.

Each hole 11 and each slot 13 includes a first dimension, called here “length,” which extends substantially parallel to the longitudinal axis B of the rod 7. Each hole 11 and each slot 13 also includes a second dimension, called here “width,” which extends around the longitudinal axis B in a plane substantially perpendicular to the axis B. Advantageously, the width of each slot 13 is substantially equal to the width of each hole 11, while the length of each slot 13 is greater than the length of each hole 11.

As shown in FIG. 4, a pin 14 connects the first part 8 and the second part 9 of the rod 7 together. The pin 14 passes through each hole 11 and each slot 13. Advantageously, the pin 14 is tightly or adjustably mounted in each hole 11.

In this configuration of the rod 7, the second end portion 7-2 of the rod 7 outside the housing 6 can include an end portion of the first part 8, with the rest of the first part 8 and the second part 9 forming the first end portion 7-1. Thus, the first part 8 is partially outside the housing 6, allowing the user of the catheter robot 1 to manipulate the rod 7 by exerting an effort on the first part 8.

The first part 8 can be manipulated to move it in translation along the axis B, in one direction or the other (advance or retreat).

As previously indicated, the length of each slot 13 is greater than the length of each hole 11. The first part 8 can therefore slide over the second part 9 during its translation along axis B without the second part 9 of the rod 7 moving together with the first part 8. Consequently, the second part 9 is decoupled in translation from the first part 8.

Since the pin 14 is tightly or adjustably mounted in each hole 11, the translation of the first part 8 over the second part 9 causes the pin 14 to move along each slot 13. The translation of the first part 8 along axis B is prevented in a given direction when the pin 14 comes into contact with one of the ends of the slot 13. In such a case, the first part 8 can be translated along axis B in the opposite direction until the pin 14 comes into contact with the other end of the slot 13.

Thanks to the sliding of the first part 8 over the second part 9, the control organ 5 can be made more compact compared to a control organ in which the entire rod 7 moves in translation along axis B.

The first part 8 can also or alternatively be manipulated to rotate around axis B, in one direction or the other (clockwise or counterclockwise).

As previously explained, the width of each slot 13 is preferably approximately equal to the width of each hole 11. Furthermore, the pin 14 is tightly or adjustably mounted in each hole 11. Thus, any rotation of the first part 8 around axis B causes the pin 14 and the second part 9 to rotate together. The second part 9 is therefore coupled in rotation with the first part 8.

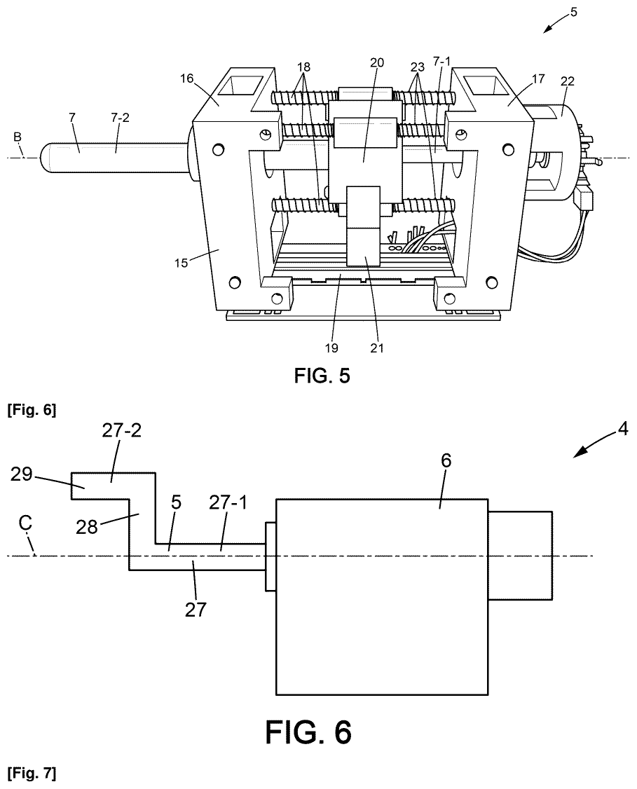

As visible in FIG. 5, the control organ 5 may further include a support 15 intended to be installed inside the housing 6 of the human-machine interface 4. The rod 7 extends partially inside the support 15. For example, the support 15 can be positioned around the rod 7 so that the end portion 7-2 outside the housing 6 is also outside the support 15.

The support 15 may include a first base 16 and a second base 17 arranged substantially perpendicularly to axis B, particularly facing each other. Advantageously, the first base 16 and the second base 17 each include a respective through hole (not visible) through which the rod 7 passes. Advantageously, the through holes of the support 15 are shaped so that the rod 7 can slide through them.

The through hole of each base 16, 17 is preferably substantially aligned with the hole of the other base 17, 16. Each hole passes through the respective base 16, 17 preferably substantially parallel to axis B. The holes in the bases 16, 17 guide the translation of the rod 7, or one of its parts, along axis B.

One or more bars 18 connect the bases 16, 17 to each other. In this case, three bars 18 are provided, but this is not limiting. Preferably, each bar 18 extends substantially parallel to axis B.

A potentiometer 19 may be provided in the control organ 5. The potentiometer 19 can be connected to the support 15. In the example of FIG. 5, the potentiometer 19 extends substantially parallel to axis B and is connected to each of the bases 16, 17 of the support 15. The potentiometer 19 is preferably of the linear type.

As shown in FIG. 5, the potentiometer 19 is connected to the rod 7. For this purpose, a pivot 20 can be arranged around the rod 7.

The pivot 20 is connected, directly or indirectly, to the potentiometer 19 so that it can slide along the potentiometer. In the example of FIG. 5, a transmission shaft 21 connects the pivot 20 and the potentiometer 19 in a sliding manner.

The pivot 20 is, for example, connected to the rod 7 by the pin 14. Advantageously, the pivot 20 is connected to the rod 7 so that it moves in translation along axis B together with the rod 7. Thus, when the rod 7 is translated along axis B, the pivot 20 follows the same movement, which varies the resistance of the potentiometer 19. The resistance of the potentiometer therefore varies according to the position of the rod 7 along axis B. In other words, the amplitude and direction of the translation along axis B of the rod 7 (or a part of it) are detected by the potentiometer 19 and translated into a variation of its resistance. Depending on the variation in resistance of the potentiometer 19, the amplitude of translation or the translation speed of the elongated flexible medical instrument 3 along its axis A varies.

It should be noted that when the rod 7 comprises two parts, only one of which is configured to be translated along axis B, such as the first part 8 in FIG. 4, the pivot 20 is connected to the part of the rod that can be translated along axis B. This allows the resistance of the potentiometer 19 to vary as soon as the rod is moved, even if only partially, along axis B.

The pivot 20 is advantageously decoupled in rotation around axis B from the rod 7. Preferably, the pivot 20 is immobile in rotation around axis B.

The control organ 5 may also include a quadrature encoder 22. The quadrature encoder 22 is connected to the rod 7. For example, the quadrature encoder 22 is connected to the end of the rod opposite the end portion 7-2 outside the housing 6 of the human-machine interface 4.

The quadrature encoder 22 is configured to measure an amplitude and direction of rotation of the rod 7 around axis B. The quadrature encoder 22 can be configured to convert the measured rotation amplitude of the rod 7 into a rotation amplitude or a rotation speed of the elongated flexible medical instrument 3 around its main elongation axis 1.

In some cases, at least one return elastic element 23, in the rest position, of the control organ 5, particularly the rod 7, may be provided. In this text, the rest position of the rod 7 refers to the position of the rod 7 before any translation movement of it, or one of its parts, along axis B. In other words, in its rest position, the rod 7 is in the position it is in before any effort is applied causing the translation movement along the longitudinal axis B of the rod 7 or one of its parts.

In FIG. 5, each return elastic element 23 is a return spring arranged around one of the bars 18 of the support 15, but this is not limiting.

As indicated, the translation movement of the rod 7 (or a part of it) along axis B or its rotation around axis B causes a translation movement of the elongated flexible medical instrument 3 along its main elongation axis A or a rotation around the main elongation axis A, respectively. Thanks to the return elastic element 23, as soon as the user of the catheter robot stops manipulating the rod 7, the rod 7 gradually moves along axis B to its rest position. As will be detailed, this can cause a gradual reduction in the movement speed of the elongated flexible medical instrument 3 to a null speed, or a retraction of the elongated flexible medical instrument 3 inside the canal in which it moves.

The human-machine interface 4 may include a safety element, allowing to block or unblock the drive of the elongated flexible medical instrument 3 by the control organ 5, in this case the rod 7.

The safety element includes a safety surface 24. In some cases, the safety surface 24 is a touch surface. In other cases, the safety surface 24 is a capacitive touch surface. In other cases, the safety surface 24 is a capacitive touch surface covered with titanium paint.

The safety surface 24 is capable of detecting the contact or pressure of the user's hand or another part of the user's body. When such contact or pressure is detected, the drive of the elongated flexible medical instrument 3 by the control organ 5 is unblocked. Thus, the translation of the rod 7 or a part of it along axis B or its rotation around axis B causes the movement of the elongated flexible medical instrument 3 in, respectively, translation along axis A or rotation around axis A. Conversely, when such contact or pressure is not detected, the drive of the elongated flexible medical instrument 3 by the control organ 5 is blocked. This prevents an involuntary movement of the rod 7, in translation and/or rotation, from triggering the movement of the elongated flexible medical instrument 3 in, respectively, translation and/or rotation.

In some cases, if no contact or pressure from the user's hand (or another part of their body) is detected by the safety surface 24, only the translation drive of the elongated flexible medical instrument 3 is blocked. Indeed, an inadvertent triggering of the translation of the elongated flexible medical instrument 3 in the patient's canal presents a greater risk to the patient's health than an inadvertent triggering of the rotation of the elongated flexible medical instrument 3, particularly when the translation is controlled in speed.

In the non-limiting example of FIG. 3, the safety surface 24 includes a first zone 24-1, a second zone 24-2, and a third zone 24-3.

The first zone 24-1 is located on an edge of the housing 6. The second zone 24-2 is located on a surface of the housing 6 that is substantially perpendicular to the first zone 24-1. Preferably, the second zone 24-2 is substantially horizontal in the usual operating position of the human-machine interface 4. The third zone 24-3 is included in the end portion 7-2 of the rod 7 outside the housing 6. In this example, the drive of the elongated flexible medical instrument 3 by the control organ 5 is unblocked when the contact or pressure of the user's hand is detected on the third zone 24-3 included in the rod 7 and on at least one of the first zone 24-1 and the second zone 24-2. Conversely, the drive of the elongated flexible medical instrument 3 is blocked when the contact or pressure of the user's hand is detected only on one of the zones 24-1 to 24-3, or on none of these zones 24-1 to 24-3.

According to a possible embodiment, the safety surface 24 is located on the rod 7, for example, by covering the rod 7 with a touch surface.

The control human-machine interface may also include haptic feedback informing the user of the catheter robot 1 of the existence of a movement of the elongated flexible medical instrument 3 inside the patient's canal. In some cases, the haptic feedback is activated only for the translation of the elongated flexible medical instrument 3. The user of the catheter robot thus receives feedback on the translation drive they have commanded, allowing them to verify that this drive is indeed taking place as they intended.

The haptic feedback is preferably in the form of vibrations. More preferably, the haptic feedback is in the form of vibrations of the control organ 5.

According to a non-limiting example, the frequency of the vibrations is proportional to the translation speed of the elongated flexible medical instrument 3. The user of the human-machine interface 4 can thus detect the passage of the elongated flexible medical instrument 3 through tortuous or stenosed areas of the canal, where the translation of the elongated flexible medical instrument 3 is hindered, thus reducing its translation speed. This allows the user to know when it is appropriate to modify or adapt the command of the translation drive of the elongated flexible medical instrument 3 and/or to command the rotation of the elongated flexible medical instrument 3.

According to another non-limiting example, the frequency of the vibrations is proportional to the translation speed of the elongated flexible medical instrument 3 when this speed is less than or equal to a threshold value. Once the translation speed of the elongated flexible medical instrument 3 exceeds this threshold value, the frequency of the vibrations remains constant regardless of the translation speed of the elongated flexible medical instrument 3. For example, the threshold value of the translation speed of the elongated flexible medical instrument 3 may be equal to 10 mm/s. Advantageously, when the translation speed of the elongated flexible medical instrument 3 is greater than the threshold value, the frequency of the vibrations is such that the user perceives a continuous vibration (i.e., the user never stops feeling the vibration). The user of the catheter robot 1 is thus alerted to a translation speed of the elongated flexible medical instrument 3 that may pose a major risk to the patient's health.

As visible in FIG. 3, the human-machine interface 4 may further include a knob 25 that can be rotated by the user's hand. As will be detailed, the rotation of the knob 25 allows the command of the movement of the additional drive module 2′ to drive the additional elongated flexible medical instrument in translation.

Since the additional drive module 2′ is controlled by the same human-machine interface 4 as the drive module 2, the overall size of the catheter robot 1 is reduced.

Preferably, the control human-machine interface also includes an additional return elastic element (not illustrated) for the knob 25 in the rest position. This additional return elastic element may include one or more additional return springs (not illustrated). In the case of the knob 25, the rest position corresponds to the position of the knob before it is rotated. As explained later, thanks to the additional return elastic element, it is possible to stop the translation drive of the additional elongated flexible medical instrument when the user stops actuating the knob 25.

The human-machine interface 4 may also include a display module 26. The display module 26 is, for example, a screen. The screen may, for example, be a touch screen, but this is not limiting.

The screen displays information about the operation of the drive module 2, and possibly the additional drive module 2′, in response to the command of these modules 2, 2′ using the control organ 5 or the knob 25, respectively.

The screen 26 may also display one or more virtual buttons allowing the selection of various functionalities of the catheter robot 1, such as an operating mode of the human-machine interface 4.

The screen 26 also allows the visualization of the position of the elongated flexible medical instrument 3, and possibly the additional elongated flexible medical instrument, in relation to the different organs of the patient. This allows the user to decide and command the various movements of the elongated flexible medical instrument during the intervention on the patient. For this purpose, an imaging system (not illustrated) can be connected to the catheter robot 1 so that the image obtained by the imaging system is visible on the screen 26.

FIG. 6 shows another embodiment of the human-machine interface 4, which differs from the human-machine interface 4 of FIG. 3 in that the control organ 5 includes a crank 27 instead of the rod 7.

The crank 27 includes a first portion 27-1 that is substantially straight along longitudinal axis C. This first portion 27-1 is similar or identical to the rod 7 and will not be described in detail hereafter.

The crank 27 further includes a second portion 27-2 located outside the housing 6 of the human-machine interface 4. The second portion 27-2 includes, for example, a first part 28 and a second part 29 fixed to each other. The first part 28 is directly connected to the first portion 27-1 of the crank 27 and extends substantially perpendicularly to the longitudinal axis C. The second part 29 extends substantially parallel to the longitudinal axis C from one end of the first part 28. The user of the catheter robot 1 can thus manipulate the crank 27, for example, with their hand, by the second part 29.

The manipulation of the crank 27 can include moving the crank 27 or a part of it in translation along the longitudinal axis C in one direction or the other (advance or retreat). The manipulation of the crank 27 can also include moving the crank 27 or a part of it in rotation around this longitudinal axis C in one direction or the other (clockwise or counterclockwise). Like the rod 7, the crank 27 is structured so that the movement of the crank 27 (or a part of it) in translation along axis C or in rotation around axis C causes a movement of the elongated flexible medical instrument 3 in, respectively, translation along its main elongation axis A or rotation around the main elongation axis A.

The remaining elements previously described with reference to the human-machine interface of FIG. 3 can also be included in the human-machine interface 4 of FIG. 6. For the sake of brevity, these elements are not described in detail again hereafter.

The embodiment illustrated in FIG. 6 allows the command of a continuous rotation movement of the elongated flexible medical instrument 3 while maintaining a position control of the rotation movement of the elongated flexible medical instrument 3.

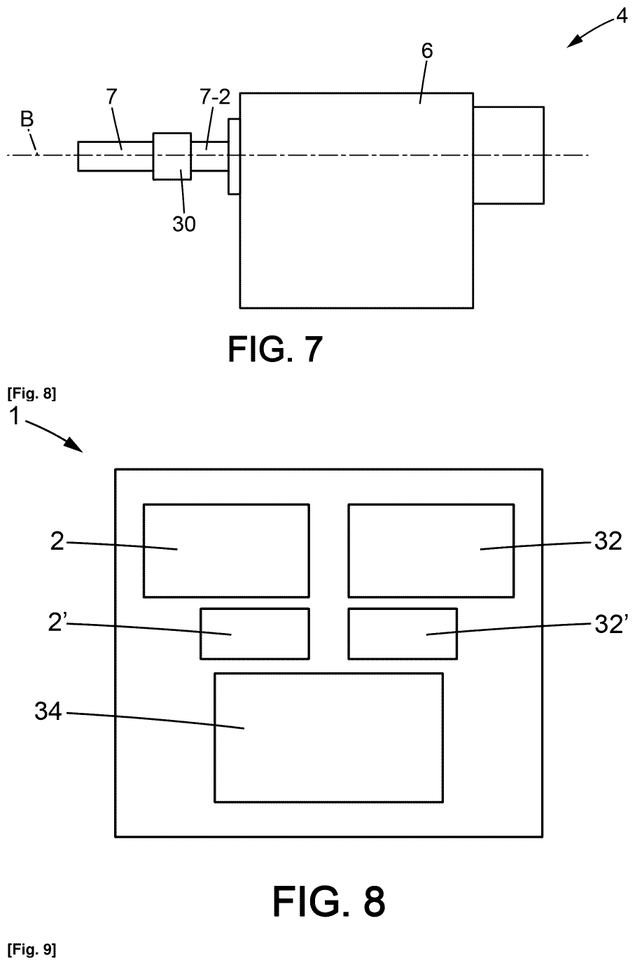

FIG. 7 shows another embodiment of the human-machine interface 4, which differs from the human-machine interface 4 of FIG. 3 in that a rotating ring 30 is located around the rod 7. In particular, the rotating ring 30 is located around the end portion 7-2 of the rod 7 outside the housing 6.

The ring 30 can be manipulated by the user of the catheter robot 1 to rotate it around the axis B of the rod 7. For example, the user can use their hand to manipulate the ring 30. Preferably, the ring 30 can be rotated around the axis B of the rod without causing the rod 7 to rotate together. As will be explained below, the rotation of the ring 30 drives the elongated flexible medical instrument 3 in rotation from one of the types of command previously indicated (in position or in speed) of a different nature from that resulting from the rotation of the rod 7 around axis B. Preferably, the ring 30 controls the rotation of the elongated flexible medical instrument 3 in speed.

A return elastic element (not illustrated), in the rest position, of the rotating ring 30 may be provided in the human-machine interface 4. The rest position of the rotating ring 30 refers to the position of the ring 30 before any rotation of the ring 30 by the user of the catheter robot 1. The return elastic element of the rotating ring 30 preferably includes a return spring.

The remaining elements previously described with reference to the human-machine interface of FIG. 3 can also be included in the human-machine interface 4 of FIG. 7. For the sake of brevity, these elements are not described in detail again hereafter.

The embodiment illustrated in FIG. 7 allows the user to control the translation of the elongated flexible medical instrument 3 in speed and the rotation of the elongated flexible medical instrument 3 in position according to a first mode of operation using only the rod 7, and to control the translation and rotation of the elongated flexible medical instrument 3 in speed according to a second mode of operation using the rod 7 for translation and the ring 30 for rotation.

Now the operation of the catheter robot 1 according to the embodiment of FIG. 1 will be described.

For the sake of brevity, in the following, when referring to the movement of the control organ 5, it includes the movement of the entire control organ 5 or a part of it (notably, the first part 8 when the control organ 5 is the rod 7 of FIG. 4).

Furthermore, in the following, when referring to the “translation movement” or “translation” of the elongated flexible medical instrument 3, it refers to the translation movement of this elongated flexible medical instrument 3 along its main elongation axis. Similarly, when referring to the “rotation movement” or “rotation” of the elongated flexible medical instrument 3, it refers to the rotation movement of the elongated flexible medical instrument 3 around its main elongation axis.

As previously indicated, the drive module 2 is connected to the elongated flexible medical instrument 3 and allows it to move in translation along axis A and/or in rotation around axis A inside a patient's canal.

Advantageously, when the control organ 5 of the human-machine interface 4 is moved, the drive module 2 moves in translation and/or rotation, causing a corresponding movement of the elongated flexible medical instrument 3 inside the patient's canal. Thus, the human-machine interface 4 is structured to control the translation and/or rotation drive of the elongated flexible medical instrument 3. As previously indicated, the control organ 5 can be moved in translation and/or rotation by the hand of the user of the catheter robot 1.

When the control organ 5 is the rod 7 or the crank 27, a translation along axis B of the rod 7 (or its first part 8 when it has the configuration of FIG. 4) or along axis C of the crank 27 causes a translation of the drive module 2 so that the elongated flexible medical instrument 3 is driven in translation along axis A. Similarly, a rotation around axis B of the rod 7 (or its first part 8 when it has the configuration of FIG. 4) or around axis C of the crank 27 causes a rotation of the drive module 2 so that the elongated flexible medical instrument 3 is driven in rotation around axis A.

Advantageously, the direction of the translation or rotation of the elongated flexible medical instrument 3 inside the patient's canal depends on the direction of movement of the control organ 5. For example, when the rod 7 (or its first part 8 when it has the configuration of FIG. 4) or the crank 27 is moved in translation in the direction of insertion into the housing 6 of the human-machine interface, the elongated flexible medical instrument 3 can advance in the patient's canal. Conversely, when the rod 7 (or its first part 8 when it has the configuration of FIG. 4) or the crank 27 is moved in translation in the direction of extraction from the housing 6 of the human-machine interface 4, the elongated flexible medical instrument 3 can retract in the patient's canal. In the case of a rotation of the rod 7 or the crank 27 in the clockwise direction, the elongated flexible medical instrument 3 can rotate around axis A in the clockwise direction, while in the case of a rotation of the rod 7 or the crank 27 in the counterclockwise direction, the elongated flexible medical instrument 3 can rotate around axis A in the counterclockwise direction.

The potentiometer 19 can be used to detect the amplitude and direction of the translation of the control organ 5. As previously indicated, the resistance of the potentiometer 19 varies according to the amplitude and direction of the translation of the control organ 5. This generates the signal to control the translation of the drive module 2, and thus, the translation of the elongated flexible medical instrument 3 inside the patient's canal. Regarding the rotation of the control organ 5, it can be detected by the quadrature encoder 22. The quadrature encoder 22 detects the amplitude and direction of the rotation of the control organ 5 and generates the control signal for the rotation of the drive module 2, and thus, of the elongated flexible medical instrument 3 inside the patient's canal.

It should be noted that when the human-machine interface 4 is equipped with the safety surface 24, the elongated flexible medical instrument 3 is only driven in translation and/or rotation when the safety surface 24 detects the contact or pressure of the user's hand, or another part of their body, as previously indicated. If such contact or pressure is not detected, the translation and/or rotation movement of the elongated flexible medical instrument 3 by the control organ 5 is blocked even if the control organ 5 is moved.

If, in a possible embodiment, the non-detection of the contact or pressure of the user's hand by the safety surface 24 only blocks the translation of the elongated flexible medical instrument 3, it is driven in rotation as soon as the control organ 5 is rotated around its axis B or axis C, even if such contact or pressure is not detected by the safety surface 24.

As also previously indicated, the translation and rotation of the elongated flexible medical instrument 3 can be controlled in position or in speed from the manipulation of the control organ 5. In the case of position control, the amplitude of movement (in translation or rotation) of the elongated flexible medical instrument 3 inside the patient's canal is proportional to the amplitude of movement (in translation or rotation) of the control organ 5. In the case of speed control, the speed of movement (in translation or rotation) of the elongated flexible medical instrument 3 inside the patient's canal is proportional to the amplitude of movement (in translation or rotation) of the control organ 5.

According to a first mode of operation, the translation drive of the elongated flexible medical instrument 3 along its axis A is controlled in speed, and the rotation drive of the elongated flexible medical instrument 3 around its axis A is controlled in position. Thus, the translation movement of the control organ 5 with a given translation amplitude causes a translation movement of the elongated flexible medical instrument 3 with a speed proportional to the translation amplitude of the control organ 5, while a rotation movement of the control organ 5 with a given rotation amplitude causes a rotation movement of the elongated flexible medical instrument 3 with a rotation angle proportional to the rotation amplitude of the control organ 5.

Generally, when the rotation of the elongated flexible medical instrument 3 is controlled in position, there is a difference between the rotation angle at the end of the elongated flexible medical instrument 3 connected to the drive module 2, called the proximal end, and the end of the elongated flexible medical instrument 3 intended to penetrate the patient, called the distal end. This is due to the fact that the distal end of the elongated flexible medical instrument 3 only starts to rotate when the proximal end of the elongated flexible medical instrument 3 has rotated by a certain angle around axis A.

To compensate for this difference between the rotation angle at the proximal end and the distal end of the elongated flexible medical instrument 3, the rotation drive of the elongated flexible medical instrument 3 can be controlled in position with a proportionality coefficient between, on the one hand, the rotation movement of the control organ 5 and, on the other hand, the rotation movement of the elongated flexible medical instrument 3. Thus, when the user of the catheter robot 1 wants to command rapid rotation movements, they can choose a high proportionality coefficient, thanks to which the rotation of the control organ 5 causes a large rotation movement of the proximal end of the elongated flexible medical instrument 3 around axis A. Conversely, if the user of the catheter robot 1 wants to command precise rotation movements, they can choose a low proportionality coefficient, thanks to which the rotation of the control organ 5 causes a small rotation movement of the proximal end of the elongated flexible medical instrument 3.

Advantageously, this proportionality coefficient can be modified by a selection made by the user of the catheter robot 1. This selection is, for example, made from a virtual button displayed on the display module 26 or from a physical control organ, such as a button, provided on the human-machine interface 4. The proportionality ratio can, for example, vary between 3/1 and 1/25. A ratio of 3/1 means that a 3° rotation of the rod 7 results in a 1° rotation of the proximal end of the elongated flexible medical instrument 3 manipulated by the drive module 2. A ratio of 3/1 can notably be used when precise movements are necessary, for example, passing a bifurcation with a guide. A ratio of 1/25 means that a 1° rotation of the rod 7 results in a 25° rotation of the proximal end of the elongated flexible medical instrument 3 manipulated by the drive module 2. A ratio of 1/25 can notably be used for a “drilling” movement (i.e., a continuous rotation movement like a screwing motion) or a “wiggling” movement (i.e., a succession of large amplitude rotation movements in the opposite direction). The proportionality ratio can vary between 3/1 and 1/20, or between 3/1 and 1/15, or between 1/1 and 1/25, or between 1/1 and 1/20, or between 1/1 and 1/15.

In this first mode of operation, thanks to the position control of the rotation of the elongated flexible medical instrument 3, the user of the catheter robot 1 can intuitively rotate the elongated flexible medical instrument 3 by a precise rotation angle around axis A. Furthermore, thanks to the speed control of the translation of the elongated flexible medical instrument, the user can move the elongated flexible medical instrument 3 over a long distance using a compact control organ 5. Additionally, the user can intuitively adjust the translation speed of the elongated flexible medical instrument 3 during its journey inside the patient's canal. This allows, for example, reducing the translation speed of the elongated flexible medical instrument 3 to cross curved areas of the canal. Conversely, when the elongated flexible medical instrument 3 crosses straight areas of the canal, the user can increase the translation speed of the elongated flexible medical instrument 3 to reach the treatment area of the canal more quickly.

In a second mode of operation, the human-machine interface 4 can be structured to control the translation drive of the elongated flexible medical instrument 3 along axis A and the rotation around axis A in speed.

In this second mode of operation, the rotation movement of the control organ 5 with a given rotation amplitude results in a rotation movement of the elongated flexible medical instrument 3 with a rotation speed proportional to the rotation amplitude of the control organ 5.

The speed control of the translation and rotation of the elongated flexible medical instrument 3 allows providing a continuous combined movement of translation and rotation of the elongated flexible medical instrument 3.

In a third mode of operation, the human-machine interface 4 can be structured to control the translation drive of the elongated flexible medical instrument 3 along axis A and the rotation around axis A in position.

In this third mode of operation, the translation movement of the control organ 5 with a given translation amplitude results in a translation movement of the elongated flexible medical instrument 3 with a translation amplitude proportional to the rotation amplitude of the control organ 5.

This third mode of operation allows obtaining precise translation and rotation amplitudes of the elongated flexible medical instrument 3. This is particularly advantageous when the elongated flexible medical instrument has reached the specific area of the patient's canal where it is used, or when passing branches as the elongated flexible medical instrument is reaching the specific area of the patient's blood system where it will be used. This third mode of operation can, for example, be used for rapid forward and backward movements of the elongated flexible medical instrument 3.

It should be noted that the human-machine interface 4 can be configured so that the user of the catheter robot 1 can choose between the first to third modes of operation of the human-machine interface 4 described above. For example, a virtual button for selecting the mode of operation can be displayed on the display module 26. This allows choosing the mode of operation best suited to the patient or the moment of the intervention.

In some cases, the human-machine interface 4 can be configured to control only the translation drive of the elongated flexible medical instrument 3 in speed and to control only the rotation drive of the elongated flexible medical instrument in position.

In each of the modes of operation of the human-machine interface 4 presented above, the translation and rotation of the elongated flexible medical instrument 3 can be controlled simultaneously or alternatively.

When the translation of the elongated flexible medical instrument 3 is controlled in position, this control can be done step by step. In such cases, the translation of the elongated flexible medical instrument 3 includes a movement of the elongated flexible medical instrument 3 along axis A by a predetermined step with each impulse received by the human-machine interface 4. Thus, the elongated flexible medical instrument 3 can, for example, make a millimetric advance or retreat in the patient's canal.

Similarly, when the rotation of the elongated flexible medical instrument 3 is controlled in position, this control can be done step by step. In such cases, the rotation of the elongated flexible medical instrument 3 includes a movement of the elongated flexible medical instrument 3 around axis A by a predetermined step with each impulse received by the human-machine interface 4. Thus, the elongated flexible medical instrument 3 can, for example, make a millimetric rotation in the patient's canal.

As previously explained, when the return elastic element 23 of the control organ 5 is provided, the control organ 5 gradually moves along its axis B or C to its rest position as soon as the user of the catheter robot stops manipulating it. Thus, thanks to the return elastic element 23, when the translation of the elongated flexible medical instrument 3 is controlled in position, the elongated flexible medical instrument 3 retracts in the patient's canal when the user stops manipulating the control organ 5. When the translation of the elongated flexible medical instrument 3 is controlled in speed, the return elastic element 23 causes a gradual reduction in the translation speed of the elongated flexible medical instrument 3 when the user stops manipulating the control organ 5, with the translation speed of the elongated flexible medical instrument 3 becoming zero if the control organ 5 returns to its rest position.

Furthermore, as in the example of FIG. 7, the human-machine interface 4 can include the rotating ring 30. When the rotating ring 30 is rotated around axis B or C, a rotation command is generated for the elongated flexible medical instrument 3. Advantageously, the rotation command generated for the elongated flexible medical instrument 3 from the rotation of the ring 30 is of a different nature from the rotation command generated by the control organ 5. For example, if the rotation of the control organ 5 controls the rotation of the elongated flexible medical instrument 3 in position, the rotation of the ring 30 controls the rotation of the elongated flexible medical instrument 3 in speed.

When the ring 30 controls the rotation of the elongated flexible medical instrument 3 in speed, the return elastic element (not illustrated), in the rest position, of the rotating ring 30 allows stopping the rotation of the elongated flexible medical instrument 3 when the user stops manipulating the ring 30.

In the case of the first mode of operation described above, the presence of the rotating ring 30 has the advantage of allowing the elongated flexible medical instrument 3 to perform a continuous and combined movement in translation and rotation respectively along and around its main elongation axis. In particular, the continuous translation movement of the elongated flexible medical instrument 3 is caused by the speed control of the translation generated by the control organ 5, while the continuous rotation movement of the elongated flexible medical instrument 3 is caused by the speed control of the rotation generated by the rotating ring 30.

As also indicated, the catheter robot 1 can include the additional drive module 2′ which is connected to the additional elongated flexible medical instrument and allows moving it in translation. In particular, when the knob 25 is turned, a translation movement command for the additional drive module 2′ is generated. The module 2′ thus moves in translation, with the additional elongated flexible medical instrument moving together with the additional module 2′.

The translation movement command generated by the rotation of the knob 25 is, for example, a speed command. In this case, thanks to the additional return elastic element of the knob 25 in the rest position, when the knob is no longer manipulated by the user, the translation speed of the additional elongated flexible medical instrument is gradually reduced.

Now the catheter robot 1 according to a second embodiment of the invention illustrated in FIG. 8 will be described.

The catheter robot 1 according to the second embodiment includes the drive module 2 for the elongated flexible medical instrument 3. The catheter robot 1 according to the second embodiment can also include the additional drive module 2′ for the additional elongated flexible medical instrument. The characteristics and operation of the drive module 2, the additional drive module 2′, the elongated flexible medical instrument 3, and the additional elongated flexible medical instrument described with reference to the first embodiment of the catheter robot 1 are applicable to this second embodiment and are not described in detail hereafter.

The catheter robot in FIG. 8 also includes a second drive module 32 for a second elongated flexible medical instrument (not illustrated). The second drive module 32 and the second elongated flexible medical instrument can be identical or similar to, respectively, the drive module 2 and the elongated flexible medical instrument 3. Therefore, they will not be described in detail hereafter.

The second drive module 32 is structured to be connectable to the second elongated flexible medical instrument. The drive module 32 is particularly configured to move the second elongated flexible medical instrument in translation along its main elongation axis, and/or in rotation around its main elongation axis.

Furthermore, the catheter robot 1 in FIG. 8 may include a second additional drive module 32′ for a second additional elongated flexible medical instrument (not illustrated). The second additional drive module 32′ and the second additional elongated flexible medical instrument can be identical or similar to, respectively, the additional drive module 2′ and the additional elongated flexible medical instrument. Therefore, they will not be described in detail hereafter.

Advantageously, the second additional drive module 32′ is structured to be connectable to the second additional elongated flexible medical instrument. The additional drive module 32′ is configured to move the second additional elongated flexible medical instrument in translation.

It should be noted that the second elongated flexible medical instrument and the second additional elongated flexible medical instrument can be introduced into a different canal of the patient than the canal into which the elongated flexible medical instrument 3 and the additional elongated flexible medical instrument are introduced. Preferably, the second elongated flexible medical instrument and the second additional elongated flexible medical instrument can be introduced into the same canal of the patient as the canal into which the elongated flexible medical instrument 3 and the additional elongated flexible medical instrument are introduced.

The catheter robot 1 in FIG. 8 also includes a human-machine interface 34.

The characteristics of the human-machine interfaces 4 described above with reference to FIGS. 3 to 7 are applicable to the human-machine interface 34 and will not be described in detail again.

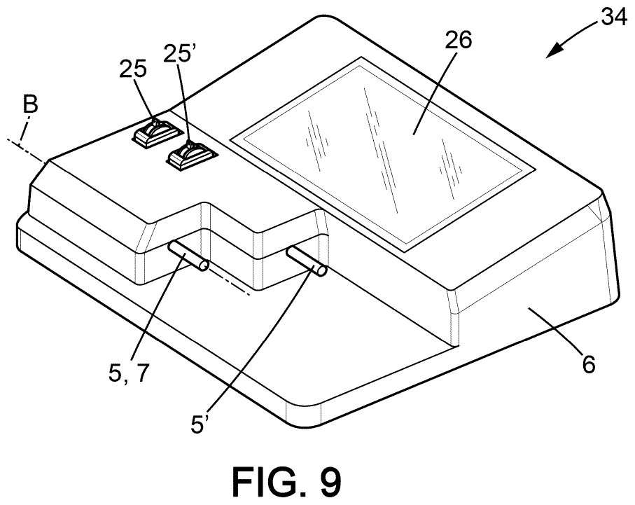

Furthermore, as clearly shown in FIG. 9, the human-machine interface 34 may include a second control organ 5′. The control organ 5′ is preferably identical or similar to the control organ 5 described above. Thus, all the characteristics of the control organ 5 indicated above are applicable to the second control organ 5′.

As will be detailed, the manipulation of the control organ 5′ drives the second elongated flexible medical instrument in translation along its main elongation axis and/or in rotation around this main elongation axis. In particular, from the control organ 5′, the translation of the second elongated flexible medical instrument along its main elongation axis can be commanded in one direction or the other (advance or retreat). Similarly, from the control organ 5′, the rotation of the second elongated flexible medical instrument around its main elongation axis can be commanded in one direction or the other (clockwise or counterclockwise). Thus, the control organ 5′ is notably a control organ for driving, in translation and rotation, the second elongated flexible medical instrument. The control organ 5′ can particularly allow the translation and/or rotation of the second elongated flexible medical instrument to be commanded from a position command or a speed command.

As visible in FIG. 9, the human-machine interface 34 may further include a second knob 25′ that can be rotated by the user's hand. The knob 25′ is preferably identical or similar to the knob 25 described above. Thus, all the characteristics of the knob 25 indicated above are applicable to the knob 25′.

As will be detailed, the rotation of the knob 25′ allows the command of the movement of the second additional drive module 32′ to drive the second additional elongated flexible medical instrument in translation.

The operation of the catheter robot 1 according to the second embodiment is similar to the operation of the catheter robot 1 according to the first embodiment. In particular, all the details of the operation of the catheter robot according to the first embodiment described above are applicable to the catheter robot 1 according to the second embodiment.

Furthermore, as indicated above, the catheter robot 1 according to the second embodiment includes the second control organ 5′. The operation of the control organ 5′ is similar to the operation of the control organ 5 described previously. Advantageously, the operation of the second control organ 5′ differs from that of the control organ 5 only in that the commands generated by the second control organ 5′ drive the movement (in translation and/or rotation) of the second drive module 32 and the second elongated flexible medical instrument. The rest of the characteristics of the operation of the control organ 5 indicated previously are therefore applicable to the control organ 5′.

It should be noted that each of the control organs 5, 5′ of the human-machine interface 34 can operate according to one of the first to third modes of operation described previously. Advantageously, the mode of operation of the control organ 5 at a given time can be the same as or different from the mode of operation of the control organ 5′. This allows adapting the way of controlling the movement of the elongated flexible medical instrument 3 and the second elongated flexible medical instrument to the particularities of the respective canals into which they are introduced.

As also indicated above, the catheter robot 1 according to the second embodiment includes the second knob 25′. The operation of the knob 25′ is similar to the operation of the knob 25 described previously. Advantageously, the operation of the knob 25′ differs from that of the knob 25 only in that the commands generated by the second knob 25′ drive the translation movement of the second additional drive module 32′ and the second additional elongated flexible medical instrument. The rest of the characteristics of the operation of the knob 25 indicated previously are therefore applicable to the knob 25′.

Of course, the present invention is not limited to the examples and embodiments described and illustrated, but it is capable of numerous variants accessible to those skilled in the art. For example, the human-machine interface 4 includes a touch surface configured to detect and measure the movement of a user's finger or a stylus along said touch surface and thus control the translation and rotation of the elongated flexible medical instrument 3. To command a translation movement of the elongated flexible medical instrument 3, the user moves their finger or the stylus along the touch surface in a first direction, the direction of the movement of the finger or stylus commanding the direction of the translation of the elongated flexible medical instrument 3, while the length of the path traveled by the finger or stylus in the first direction commands the translation speed of the elongated flexible medical instrument 3. To command a rotation movement of the elongated flexible medical instrument 3, the user moves their finger or the stylus along the touch surface in a second direction perpendicular to the first direction, the direction of the movement of the finger or stylus commanding the direction of the rotation of the elongated flexible medical instrument 3, while the length of the path traveled by the finger or stylus in the second direction commands the angular position of the elongated flexible medical instrument 3 with a ratio of x mm=y° (i.e., x mm of movement along the second direction results in a y° rotation of the elongated flexible medical instrument 3). The user can command a combined translation and rotation movement of the elongated flexible medical instrument 3 by moving their finger or the stylus along the touch surface in a third direction that includes a component in the first direction and a component in the second direction.

1-21. (canceled)

22. A catheter robot comprising:
a drive module for an elongated flexible medical instrument, in translation along a main elongation axis of said elongated flexible medical instrument and in rotation around the main elongation axis of said elongated flexible medical instrument, simultaneously or alternatively,
a human-machine interface for controlling said drive module,
wherein said human-machine interface is structured to, in a first mode of operation:
control, in speed, the translation drive of said elongated flexible medical instrument,
control, in position, the rotation drive of said elongated flexible medical instrument.

23. The catheter robot according to claim 22, wherein said human-machine interface comprises:
a mobile control organ:
intended to be manipulated by the hand of a user,
and structured so that, in the first mode of operation:
a translation movement of said mobile control organ with a translation amplitude causes a translation movement of said elongated flexible medical instrument with a speed proportional to said translation amplitude,
a rotation movement of said mobile control organ with a rotation amplitude causes a rotation movement of said elongated flexible medical instrument with a rotation angle proportional to said rotation amplitude.

24. The catheter robot according to claim 22, wherein:
said human-machine interface is structured to:
control the rotation drive of said elongated flexible medical instrument in position, with a proportionality coefficient between the rotation movement of said control organ and the rotation movement of said elongated flexible medical instrument,
said proportionality coefficient being adjustable by a selection made by the user of the catheter robot.

25. The catheter robot according to claim 22, wherein:
said human-machine interface for control comprises:
a control organ for driving, in translation and rotation, said elongated flexible medical instrument,
a safety element, allowing to block or unblock the drive of said elongated flexible medical instrument by said control organ for driving.

26. The catheter robot according to claim 25, wherein:
said safety element comprises a safety surface capable of detecting the contact or pressure of the user's hand to unblock said control organ for driving,
said safety surface being preferably a touch surface, or a capacitive touch surface, or a capacitive touch surface covered with a coating including titanium, or a capacitive touch surface covered with titanium paint.

27. The catheter robot according to claim 22, wherein:
said human-machine interface for control includes:
haptic feedback for only the translation of the elongated flexible medical instrument,
preferably in the form of vibrations,
or preferably in the form of vibrations whose frequency is proportional to the translation speed of the elongated flexible medical instrument.

28. The catheter robot according to claim 22, wherein the human-machine interface is structured to, in a second mode of operation:
control the translation drive of said elongated flexible medical instrument in speed,
control the rotation drive of said elongated flexible medical instrument in speed.

29. The catheter robot according to claim 22, wherein:
said human-machine interface for control includes:
a rod:
which is translatable by the hand of a user to drive said elongated flexible medical instrument in translation,
which is rotatable by the hand of a user to drive said elongated flexible medical instrument in rotation,
and which preferably includes a touch surface.

30. The catheter robot according to claim 29, wherein:
said rod comprises two parts sliding into each other:
a first part that is mobile in translation and rotation and is intended to be manipulated by the hand of a user,
a second part that is coupled in rotation with said first part, and that is decoupled in translation from said first part to remain fixed in translation.

31. The catheter robot according to claim 29, wherein:
said human-machine interface also includes:
a rotating ring disposed around said rod, the rotation angle of said rotating ring around said rod being representative of the selected rotation speed for said elongated flexible medical instrument, during a speed control of the rotation drive of said elongated flexible medical instrument.

32. The catheter robot according to claim 30, wherein:
said human-machine interface also includes:
a return elastic element, in the rest position, of said rotating ring disposed around said rod, the return elastic element preferably comprising a return spring.

33. The catheter robot according to claim 22, wherein:
said human-machine interface for control includes:
a crank:
which is rotatable by the hand of a user to drive said elongated flexible medical instrument in rotation.

34. The catheter robot according to claim 23, wherein:
said human-machine interface for control also includes:
a return elastic element, in the rest position, for the translation drive only, of said mobile control organ or said rod, said return elastic element preferably comprising a return spring.

35. The catheter robot according to claim 22, further comprising:
another drive module for another elongated flexible medical instrument, in translation along a main elongation axis of said other elongated flexible medical instrument and in rotation around the main elongation axis of said other elongated flexible medical instrument,
said human-machine interface also controlling said other drive module and being structured to, in the first mode of operation:
control, in speed, the translation drive of said other elongated flexible medical instrument,
control, in position, the rotation drive of said other elongated flexible medical instrument.

36. The catheter robot according to claim 22, further comprising:
an additional drive module, in translation, for an additional elongated flexible medical instrument, said additional elongated flexible medical instrument surrounding said elongated flexible medical instrument over part of its length,
in that:
said human-machine interface also controls said additional drive module, and in that:
said human-machine interface is structured to control the translation drive of said additional elongated flexible medical instrument in speed.

37. The catheter robot according to claim 35, wherein:
said human-machine interface for control also includes:
a knob that is rotatable by the hand of a user to control in speed and drive in translation said additional elongated flexible medical instrument.

38. The catheter robot according to claim 37, wherein:
said human-machine interface for control also includes:
an additional return elastic element for the knob in the rest position, which preferably comprises one or more additional return springs.

39. The catheter robot according to claim 22, wherein said human-machine interface is structured to:
control, only in speed, the translation drive of said elongated flexible medical instrument,
control, only in position, the rotation drive of said elongated flexible medical instrument.

40. The catheter robot according to claim 22, wherein:
said human-machine interface is structured, in a third mode of operation, to:
control the translation drive of said elongated flexible medical instrument in position, step by step, i.e., by moving a predetermined step with each impulse received by the human-machine interface,
control the rotation drive of said elongated flexible medical instrument in position, step by step, i.e., by moving a predetermined step with each impulse received by the human-machine interface.

41. A human-machine interface for controlling a drive module of an elongated flexible medical instrument, in translation along a main elongation axis of said elongated flexible medical instrument and in rotation around the main elongation axis of said elongated flexible medical instrument, in a catheter robot, wherein it is structured to, in a first mode of operation:
control the translation drive of said elongated flexible medical instrument in speed,
control the rotation drive of said elongated flexible medical instrument in position.

42. The human-machine interface according to claim 41, further comprising:
a mobile control organ:
intended to be manipulated by the hand of a user,
and structured so that, in the first mode of operation:
a translation movement of said mobile control organ with a translation amplitude causes a translation movement of said elongated flexible medical instrument with a speed proportional to said translation amplitude,
a rotation movement of said mobile control organ with a rotation amplitude causes a rotation movement of said elongated flexible medical instrument with a rotation angle proportional to said rotation amplitude.
